(19) United States
(12) Patent Application Publication
(10) Pub. No.: US 2009/0149418 A1
(43) Pub. Date: Jun. 11, 2009

(54) Antimicrobial preparations using polysaccharide gel from durian fruit-rind

(76) Inventors: Sunanta Pongsamart, Patumwan (TH); Vimolmas Lipipun, Patumwan (TH)

Correspondence Address: Dr. Sunanta Pongsamart;Department of Biochemistry, Faculty of Pharmaceutical Sciences, Chulalongkorn University, Phyathai Rd., Patumwan, Bangkok, 10330, TH

(21) Appl. No.: 11/999,865

(22) Filed: Dec. 7, 2007

(51) Int. Cl. A61K 31/715; A61P 31/00

(52) U.S. Cl. 514/54

(57) ABSTRACT

A water-soluble polysaccharide gel having good gelling property and good antimicrobial activity was extracted from fruit-rind of durian (Durio zibethinus Murr.) comprises steps of boiling dried fruit rind with 25-30 volumes by weight of water, filtering, repeating boiling residue 1-2 times with 15 volume by weight of water. Filtrates were pooled and concentrated by rotary evaporating at 70° C. under reduced pressure and precipitating using acidified aqueous ethanol, collecting precipitate, spray-drying or drying and pulverizing. Pharmaceutical preparations were prepared using the polysaccharide gel such as anti-acne preparations, alcohol-free hand sanitizer preparation and wound antibacterial gel preparation.

### BACKGROUND OF THE INVENTION

This invention relates to antibacterial preparation to be applied on skin prepared from polysaccharide gel extracted from durian rind.

Recent research on applications of natural polysaccharide gel has received great attention because these compounds are of high hydrolytic stability, yet are biodegradable. The natural polysaccharides isolated from dried fruit-rind of durian are high-molecular weight polysaccharides present in fruit-rind of durian.

Polysaccharides are usually defined as polymers from plant cell wall that are soluble in water. It has great industrial potential due to its useful physical and chemical properties with advantageous biological activities. Potential future applications of polysaccharides are food additives, thickeners, stabilizer, emulsifiers, gelling agents, adhesives, and adsorbents. Most of polysaccharide gel fraction, obtained from hot-water extraction and precipitation in acid-alcohol, is therefore able to disperse and swollen in water. Efficient separation of these plant components will promote biomass utilization as valuable industrial raw materials to derive wide range of products for medical, pharmaceutical, cosmetics and food industries.

### SUMMARY OF THE INVENTION

A water-soluble polysaccharide gel was extracted from fruit-rind of durian (Durio zibethinus Murr.) comprises steps of mild boiling dried fruit rind in 25-30 volume of water for 40 minutes, filtering, repeating boiling residue 1-2 times with 15 volume by weight of water. Filtrates were pooled and concentrated by rotary evaporating at 70° C. under reduced pressure and precipitating using acidified aqueous ethanol, collecting precipitate, and spray-drying or drying and pulverizing. The polysaccharide gel has very good gelling property including good antibacterial activity against various pathogenic microorganisms. Pharmaceutical preparations were prepared using the polysaccharide gel such as anti-acne preparations, alcohol-free hand sanitizer preparation and wound antibacterial gel preparation.

### DETAILED DESCRIPTION OF THE PREFERRED EMBODIMENTS

Polysaccharide gel was extracted from rind of durian (Durio zibethinus Murr.) a plant cultivated in Thailand with delicious fruit (Pongsamart and Panmaung (1998) Songkhlanakarin J Sci Technol, 20 (3), 323-332). The gel itself was found to possess good antibacterial activity against various microorganisms i.e. Staphylococcus aureus, Staphylococcus epidermidis, Micrococcus luteus, Bacillus subtilis, Escherichia coli, and Lactobacillus pentosus (Pongsamart et al (2005), Acta Horticulturae 678, 65-73). This polysaccharide gel is water-soluble and has good physical property as it becomes gel readily and turns liquefied instantaneously upon shaking which is very desirable to be used pharmaceutically as suspending agent, gelling agent and thickening agent, in addition to its desirable adhesive property. Table 1 shows antibacterial activity of polysaccharide gel against those various strains of bacteria including Propionibacterium acnes and Proteus vulgaris. PG preparation was shown to heal full thickness of excised wound on pig skin (Table 2). This invention describes antibacterial preparations comprises mainly polysaccharide gel from Durian fruit-rind prepared as antiseptic gel and lotions, anti-acne product, alcohol-free hand sanitizer, wounds antibacterial gel and more.

Durian fruits are harvested and fruit-rind is dried and used. Polysaccharide gel is extracted from dried fruit-rind of durian by boiling in water for a predetermined period of time. One kilogram of chopped fresh fruit-rind is dried in hot air oven at 50-60° C. for 20-24 hr, a total of two hundred grams of dried fruit-rind is obtained. Dried residue is suspended in 25-30 volume by weight of hot distilled water and heated to mild boiling for 40 minutes and filtered. The process is repeated 1-2 times with 15 volume by weight of boiling water, and filtrates are pooled. Water extract of polysaccharide gel is concentrated by rotary evaporating at 70° C. under reduced pressure and precipitated using acidified aqueous ethanol. Precipitate is collected, dried and pulverized according to Pongsamart and Panmaung (1998) or spray-drying to obtain dry pale beige-color powder of polysaccharide gel designated herein as “PG”. PG is used to prepare antibacterial preparations either anti-acne preparation or antibacterial gel, etc.

### EXAMPLE 1

Antimicrobial Preparation

| | |
|---|---|
| PG | 2-5% w/v |
| Lactic acid | 0-1% w/v |
| Salicylic acid | 0-1% w/v |
| Surface active agent(s) | 0-20% w/v |
| Adjuvant antimicrobial agent(s) | 0-20% w/v |
| Emollient | 0-10% w/v |
| Co-solvent(s) | 5-20% w/v |
| Triethanolamine | 0-1% w/v |
| Preservative(s) | 0-1% w/v |
| Water or physiological solution | q.s. ad |

PG is dispersed in distilled water while stirring to allow full swelling to obtain homogeneous solution. Lactic acid or salicylic acid or any appropriate keratolytic agent and betel vine oil and/or tea tree oil or any appropriate antimicrobial agent is solubilized in co-solvent or surfactant and added to PG solution. In case oil-soluble antimicrobial agent is chosen to use in the preparation, a surface active agent is used to help mixing with PG solution.

Co-solvent is chosen from a group comprising glycerin, sorbital, propylene glycol, and polyethylene glycol (PEG) of different molecular weights.

### EXAMPLE 2

Anti-acne Gel Preparation No. 1

| | | |
|---|---|---|
| PG | 2.5% | w/v |
| Lactic acid | 0.5% | w/v |
| Betel vine oil | 1.0% | w/v |
| Caster oil derivative | 5.0% | w/v |
| Lanolin derivative | 0.25% | w/v |
| Glycerin and/or Propylene Glycol | 5.0% | w/v |
| Triethanolamine | 0-1% | w/v |
| Preservative(s) | 0-1% | w/v |

| | |
|---|---|
| Water | q.s. ad |

### EXAMPLE 3

Anti-acne Gel Preparation No. 2

| | | |
|---|---|---|
| PG | 2.5% | w/v |
| Salicylic acid | 0.5% | w/v |
| Betel vine oil | 1.0% | w/v |
| Caster oil derivative | 5.0% | w/v |
| Lanolin derivative | 0.25% | w/v |
| Glycerin and/or Propylene Glycol | 5.0% | w/v |
| Triethanolamine | 0-1% | w/v |
| Preservative(s) | 0-1% | w/v |

| | |
|---|---|
| Water | q.s. ad |

### EXAMPLE 4

Anti-acne Gel Preparation No. 3

| | | |
|---|---|---|
| PG | 2.5% | w/v |
| Betel vine oil | 1-2% | w/v |
| Caster oil derivative | 5-10% | w/v |
| Lanolin derivative | 0.25-0.5% | w/v |
| Glycerin and/or Propylene Glycol | 5.0% | w/v |
| Triethanolamine | 0-1% | w/v |
| Preservative(s) | 0-1% | w/v |

| | |
|---|---|
| Water | q.s. ad |

### EXAMPLE 5

Alcohol-free Hand Sanitizer Preparation

| | | |
|---|---|---|
| PG | 2-2.5% | w/v |
| Tea-tree oil | 1.0% | w/v |
| Betel vine oil | 0.2-0.5% | w/v |
| Menthol | 1.2% | w/v |
| Caster oil derivative | 5-15% | w/v |
| Lanolin derivative | 0.5% | w/v |
| Propylene glycol | 10-15% | w/v |
| Triethanolamine | 0-1% | w/v |
| Preservative(s) | 0-1% | w/v |

| | |
|---|---|
| Water | q.s. ad |

### EXAMPLE 6

Wound Antibacterial Gel Preparation

| | | |
|---|---|---|
| PG | 2.5% | w/v |
| Glycerin | 5-10% | w/v |
| Propylene glycol | 5-10% | w/v |
| Preservative(s) | 0-1% | w/v |

| | |
|---|---|
| Water or Physiological saline solution | q.s. ad |

Anti-acne Gel Preparation No. 1, No. 2 and No. 3 were tested against Propionibacterium acnes, Staphylococcus aureus, and Staphylococcus epidermidis and found to have good antibacterial activity. Alcohol-free hand sanitizer was tested against Staphylococcus aureus, Staphylococcus epidermidis, Bacillus subtilis, Micrococcus luteus, Escherichia coli, Proteus vulgaris, Salmonella typhimurium, Krebsiella pneumoniae, Pseudomonas aeruginosa and Candida albicans. The preparation was found to have good antimicrobial activity.

It will be understood that modifications can be made in the above description without departing from the scope of this invention by one of ordinary skill in the art. It is accordingly intended that all matter contained in the above description be interpreted as descriptive and illustrative rather than in a limiting sense. It is also to be understood that the following claims are intended to cover all of the generic and specific features of the invention as described herein, and all statements of the scope of the invention which, as a matter of language, might be said to fall therebetween.

We claim

1. A water-soluble polysaccharide gel to be used as antimicrobial agent where it is extracted from dried fruit-rind of durian comprises steps of mild boiling dried fruit rind in 25-30 volume of water for 40 minutes, filtering, repeating mild boiling residue 1-2 times with 15 volume by weight of water, pooling of filtrates and concentrating by rotary evaporating at 70° C. under reduced pressure and precipitating using acidified aqueous ethanol, collecting precipitate, spray-drying or drying and pulverizing.

2. An antimicrobial preparation using polysaccharide gel of claim 1. comprises

| | |
|---|---|
| Polysaccharide gel | 2-5% w/v |
| Lactic acid | 0-1% w/v |
| Salicylic acid | 0-1% w/v |
| Adjuvant antimicrobial agent(s) | 0-20% w/v |
| Surface active agent(s) | 0-20% w/v |
| Emollient | 0-10% w/v |
| Co-solvent(s) | 5-20% w/v |
| Triethanolamine | 0-1% w/v |
| Preservative(s) | 0-1% w/v |
| Water or physiological solution | q.s. ad. |

3. Anti-acne gel preparation using polysaccharide gel of claim 1. comprises

| | | |
|---|---|---|
| Polysaccharide gel | 2.5% | w/v |
| Lactic acid | 0.5% | w/v |
| Betel vine oil | 1.0% | w/v |
| Caster oil derivative | 5.0% | w/v |
| Lanolin derivative | 0.25% | w/v |
| Glycerin and/or propylene glycol | 5.0% | w/v |
| Triethanolamine | 0-1% | w/v |
| Preservative(s) | 0-1% | w/v |
| | |
|---|---|
| Water | q.s. ad. |

4. Anti-acne gel preparation using polysaccharide gel of claim 1. comprises

| | | |
|---|---|---|
| Polysaccharide gel | 2.5% | w/v |
| Salicylic acid | 0.5% | w/v |
| Betel vine oil | 1.0% | w/v |
| Caster oil derivative | 5.0% | w/v |
| Lanolin derivative | 0.25% | w/v |
| Glycerin and/or propylene glycol | 5.0% | w/v |
| Triethanolamine | 0-1% | w/v |
| Preservative(s) | 0-1% | w/v |
| | |
|---|---|
| Water | q.s. ad. |

5. Anti-acne gel preparation using polysaccharide gel of claim 1. comprises

| | | |
|---|---|---|
| Polysaccharide gel | 2.5% | w/v |
| Betel vine oil | 1-2% | w/v |
| Caster oil derivative | 5-10% | w/v |
| Lanolin derivative | 0.25-0.5% | w/v |
| Glycerin and/or Propylene Glycol | 5.0% | w/v |
| Triethanolamine | 0-1% | w/v |
| Preservative(s) | 0-1% | w/v |
| | |
|---|---|
| Water | q.s. ad |

6. Alcohol-free hand sanitizer preparation using polysaccharide gel of claim 1. comprises

| | | |
|---|---|---|
| Polysaccharide gel | 2-2.5% | w/v |
| Tea Tree Oil | 1.0% | w/v |
| Betel Vine Oil | 0.2-0.5% | w/v |
| Menthol | 1.2% | w/v |
| Caster oil derivative | 5-15% | w/v |
| Lanolin derivative | 0.5% | w/v |
| Propylene Glycol | 10-15% | w/v |
| Triethanolamine | 0.1% | w/v |
| Preservative(s) | 0-1% | w/v |
| | |
|---|---|
| Water | q.s. ad |

7. Wound Antibacterial Gel Preparation using polysaccharide gel of claim 1. comprises

| | | |
|---|---|---|
| Polysaccharide gel | 2.5% | w/v |
| Glycerin | 5-10% | w/v |
| Propylene glycol | 5-10% | w/v |
| Preservative(s) | 0-1% | w/v |
| | |
|---|---|
| Physiological saline solution | q.s. ad |
